(19) United States
(12) Patent Application Publication
(10) Pub. No.: US 2025/0078959 A1
(43) Pub. Date: Mar. 6, 2025

(54) SYSTEM AND METHOD FOR PREDICTING MONOMERIC HEAD AND TAIL POSITIONS IN POLYMERIZATION AND RETROSYNTHESIS

(71) Applicant: International Business Machines Corporation, Armonk, NY (US)

(72) Inventors: Ronaldo GIRO, Sao Paulo (BR); Mathias B. STEINER, Rio de Janeiro (BR); Brenda DE SOUZA FERRARI, Espirito Santo (BR)

(21) Appl. No.: 18/457,592

(22) Filed: Aug. 29, 2023

(51) Int. Cl. G16C 10/00; G16C 20/30; G16C 20/70

(52) U.S. Cl. CPC G16C 10/00; G16C 20/30; G16C 20/70

(57) ABSTRACT

Method and apparatus for assigning a head and tail of a monomer. An input is obtained, where the input includes a simplified molecular input line entry. A quantum mechanics tool is generated based on the simplified molecular line entry. An atomic population of a monomer is extracted using the quantum mechanics tool. A functional group of the monomer is identified. A polymerization based on the atomic population of the monomer and the functional group is determined. A head and a tail of the polymerization site of the monomer is assigned.

### BACKGROUND

The present disclosure relates to cheminformatics, and more specifically, to polymerization reactions.

A monomer is a molecule that can react together with other monomer molecules to form a polymer using two or more atoms of the monomer. The respective locations of the two or more atoms of the monomer designate a head and a tail of the monomer. Polymer properties, such as glass transition temperature, refractive index, and tensile strength, will be based on how the two monomers are linked. The head and the tail of the monomer is where the polymerization reaction will occur. For example, a polymer produced using benzene monomers may polymerize at a para, ortho, or meta position. A polymerization at the ortho or meta position will form a non-linear, or branched, polymer, while a polymerization at the para position will form a linear polymer. The linear polymer and the branched polymer will result in different polymer properties. As such, the head and tail of the monomer will define the material field of application for the polymer after polymerization.

Generally, the properties of the polymer may be determined prior to polymerization by simulating the properties according to one or more models. In this way, determine the head and tail atom positions from the monomer is of importance. Although some tools propose to directly or indirectly identify these positions, this problem is not yet resolved. For example, a program known as “OPSIN” (Open Parser for Systematic IUPAC Nomenclature) was developed to provide a simplified molecular input line entry system (SMILES) for polymers. Unfortunately, OPSIN requires the use of IUPAC names of polymers, in which an unknown polymer could not be entered in the model as the program operates by parsing the chemical name of the monomer from left to right. Alternatively, model known as “m2p” (monomers to polymers) was developed to convert monomers to polymers using SMILES representations. However, this model relies solely on the SMILES representation of oligomers and does not identify the head and tail of the monomer. In addition, the polymerization may be inaccurate in cases with two or more functional groups since there is no condition to establish the priority of a functional group due to relative nucleophilicity not being related to head and tail assignment. Additionally, the model accounted for a low number of polymerization classes which limits the usage of the program.

Accordingly, a computer-implemented method of identifying a head and tail of a monomer is necessitated.

### SUMMARY

According to one embodiment of the present disclosure, a computer implemented method is disclosed. The computer implemented method includes obtaining an input. The input includes a simplified molecular input line entry (SMILE). The computer implemented method includes generating a quantum mechanics tool based on the simplified molecular line entry. The computer implemented method includes extracting an atomic population of a monomer using the quantum mechanics tool. The computer implemented method includes identifying a functional group of the monomer. The computer implemented method includes determining a polymerization site based on the atomic population of the monomer and the functional group. The computer implemented method includes assigning a head and tail of the polymerization site of the monomer.

According to another embodiment of the present disclosure, a system is disclosed. The system includes a computer, a polymerization dataset, a network, and a server. The server is configured to obtain an input. The input includes a simplified molecular input line entry. The server is configured to generate a quantum mechanics tool based on the simplified molecular line entry. The server is configured to extract an atomic population of a monomer using the quantum mechanics tool. The server is configured to identify a functional group of the monomer. The server is configured to determine a polymerization site based on the atomic population of the monomer and the functional group. The server is configured to assign a head and tail of the polymerization site of the monomer.

According to another embodiment of the present disclosure, a computer program product for assigning a head and tail of a monomer is disclosed. The computer program product includes a computer-readable storage medium having computer-readable program code embodied therewith. The computer program product code is executable by one or more computer processors to obtain an input, where the input comprises a simplified molecular input line entry, generate a quantum mechanics tool based on the simplified molecular line entry, extract an atomic population of a monomer using the quantum mechanics tool; identify a functional group of the monomer, determine a polymerization site based on the atomic population of the monomer and the functional group; and assign a head and tail of the polymerization site of the monomer.

### BRIEF DESCRIPTION OF THE DRAWINGS

FIG. 1 shows a computing environment, according to aspects of the present disclosure.

FIG. 2 shows a representation of a monomer, pseudo repeating unit, repeating unit, and a head and tail of a repeating unit, according to embodiments of the present disclosure.

FIG. 3 shows a system workflow, according to aspects of the present disclosure.

FIG. 4 shows a computer-implemented method workflow, according to aspects of the present disclosure.

FIGS. 5A and 5B show an atomic population of a monomer and a nucleophilicity ranking of the atoms belonging to functional groups, according to aspects of the present disclosure. FIG. 5A shows an atomic population of a monomer. FIG. 5B shows a nucleophilicity index ranking of the atoms belonging to functional groups based on the atomic population of the monomer.

FIGS. 6A and 6B show a computer-implemented method workflow of determining a p-hydroxystyrene monomer, according to aspects of the present disclosure. FIG. 6A shows a method of determining a p-hydroxystyrene monomer using chemical similarity. FIG. 6B shows a method of assigning a head and tail to the p-hydroxystyrene monomer.

FIGS. 7A and 7B show a validation of the performance of the head and tail assignment engine, according to embodiments of the disclosure. FIG. 7A shows a validation of the performance of the head and tail assignment engine in determining a polymer class. FIG. 7B shows a validation of the performance of the head and tail assignment engine in assigning the head and tail positions.

### DETAILED DESCRIPTION

In the following, reference is made to embodiments presented in this disclosure. However, the scope of the present disclosure is not limited to specific described embodiments. Instead, any combination of the following features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the following aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

In an aspect, the present disclosure provides a computer-implemented method of identifying a head and tail of a monomer such that accurate polymer properties are capable of being predicted. Any monomer can be utilized, e.g., an IUPAC named monomer, an unknown monomer, or unnamed monomer, allowing for a wide range of monomers to be utilized, as the only requirement of the monomer is the simplified molecular input line entry system (SMILES) format, allowing for a broad range of compounds to be used. Using an atomic population of the monomer allows for a nucleophilicity index of each atom of the monomer to be determined, which creates improved identification of the head and tail of the monomer, allowing for improved polymer property predictions. In an embodiment, the atoms can be ranked using the nucleophilicity index, allowing for the head and tail of a monomer having two or more functional groups to be identified, which reduces overall error in modeling of the polymer. By incorporating head and tail prediction on a pipeline study of polymers, a reduction in a large number of unnecessary chemical syntheses is achieved, which reduces the overall cost of production as well as time spent on identifying target polymers.

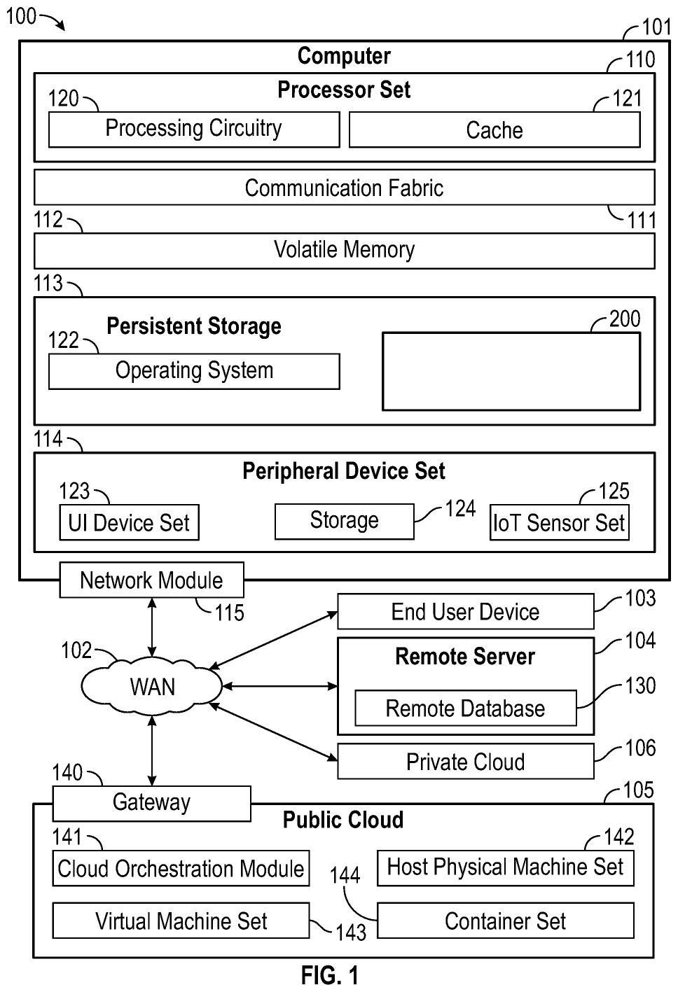

With reference now to FIG. 1, various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as Python script tool to assign head and tail atom positions in a monomer defining the polymer repeat unit 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

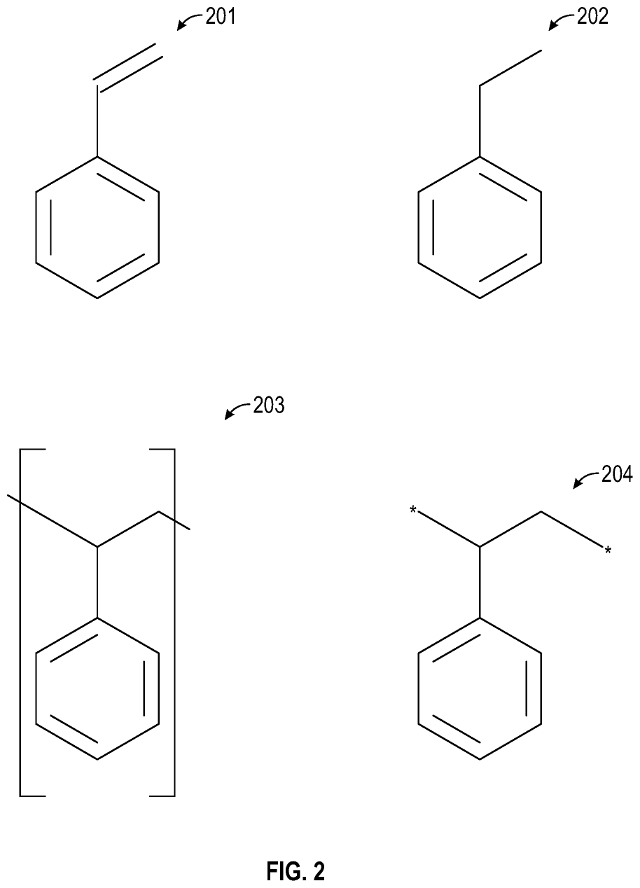

Now referring to FIG. 2, a monomer 201, pseudo repeating unit 202, repeating unit 203, and a head and tail tagged repeating unit 204 is shown. A “monomer,” as used herein, is a molecule that can react with one or more other monomer molecules to form a larger polymer chain or three dimensional molecule in a process known as polymerization. A monomer 201 may be subdivided into two classes, monomers that participate in step polymerization or monomers that participate in chain polymerization. A monomer 201 can be natural or synthetic. For example, a natural monomer can include monomeric proteins, amino acids such as glycine, nucleotides, carbohydrates such as glucose, fructose, sucrose, or any other compound having a glyosidic bond, or isoprene. For example, a synthetic monomer can include ethylene, tetrafluoroethylene, vinyl chloride, styrene, epoxides, bisphenol A, terephthalic acid, dimethyl silicon dichloride, ethyl methacrylate. In an embodiment, the monomer can be styrene. A monomer 201 can be polar or non-polar, e.g., a polar monomer can be vinyl acetate, whereas a nonpolar monomer can be ethylene. A monomer 201 can be cyclic or linear, e.g., a cyclic monomer can be ethylene oxide, whereas a linear monomer can be ethylene glycol. Without being bound by theory, the structure of the monomer, once identified, allows for determining the atomic population of the atoms present on the monomer, using computer 101, described herein. The atomic population can then be used to rank a nucleophilicity index of the atoms of the monomer to determine a predicted polymerization binding scheme using computer 101 described herein, in which a binding scheme is a head-to-tail, tail-to-tail, or tail-to-head binding pattern. Additionally, the computer 100 can identify the head and tail of the polymerization location.

A monomer 201 may be characterized by a pseudo repeating unit 202. A “pseudo repeating unit,” as used herein, is a representation of the monomer that will be present in the polymer, in which the bonds that are formed as a result of the polymerization are not shown. For example, styrene may have a pseudo repeating unit 202 represented by a single carbon bond where the vinyl group is present in the monomer. The pseudo repeating unit 202 may assist in representing the branches of the polymer that will form using the monomer.

A monomer 201 may be characterized by a repeating unit 203 once the monomer 201 has been polymerized. A “repeating unit,” as used herein, is a representation of a monomer that depicts the part of the monomer that would repeat to produce the complete polymer chain by linking the repeat units together successively along the chain. For example, a repeating unit 203 can include a —[CH₂—CH₂]— unit for the addition polymer polyvinyl chloride —[CH₂—CH₂]n-. The repeating units can replace the C═C double bond in the monomer by a C—C single bond, which links two new bonds to adjoining repeat units formed by one or more other monomers. As a further example, and still referring to FIG. 2, the repeating unit 203 of styrene may remove the vinyl group in favor of a C—C bond, which includes two additional bonds that are severed by brackets indicating that the unit repeats in a larger polymer. Alternatively, a repeating unit 203 may contain fewer atoms than the monomer or monomers from which it is formed where the monomer is used for a condensation polymerization.

A monomer 201 may be characterized by a head and tail tagged repeating unit 204. The head and tail are represented by atoms of the repeating unit that are tagged with a notation, e.g., a star, radical, etc., that indicates which atoms will be the site of polymerization. The head tag indicates the atom that belongs to a functional group and has a higher nucleophilicity index, e.g., is the nucleophile for the polymerization reaction of the monomer and/or a free radical of a polymerizations reaction of the monomer. The tail tag indicates the atom that belongs to the same functional group as the head and has a lower nucleophilicity index, e.g., is the electrophile for the polymerization reaction of the monomer. In an embodiment, a monomer having an atom tagged as the head may interact with an alternative monomer having an atom tagged as the tail, where a polymerization reaction occurs between the head and the tail. In another embodiment, a polymerization reaction may occur to form atoms connections of head-to-head and tail-to-tail. For example, a monomer having an atom tagged as the head may interact with an alternative monomer having an atom tagged as the head, where a polymerization reaction occurs between the head of the monomer and the head of the alternative monomer. As a further example, a monomer having an atom tagged as the tail may interact with an alternative monomer having an atom tagged as the tail, where a polymerization reaction occurs between the tail of the monomer and the tail of the alternative monomer. For example, and still referring to FIG. 2, styrene may have an atom tagged as a head that is located at a distal carbon of the ethyl group, where the tail may be tagged as the proximal carbon of the ethyl group. The tagging of the head and tail allows for ranking the atoms based on nucleophilicity index to determine a predicted polymerization binding scheme.

A monomer 201 may include one or more functional groups. A “functional group,” as used herein, is an atom or group of atoms within a monomer that has similar chemical properties whenever it appears in various compounds, in which the functional group may be a location or situs for a chemical reaction. A functional group can include alkenes and/or alkynes. Alkenes are hydrocarbons that contain one or more double bonds between neighboring carbons, whereas alkynes contain one or more triple bonds between neighboring carbons. Without being bound by theory, atoms of the alkenes and alkynes may be the situs for one or more addition polymerization reactions. A functional group can include an aromatic. An aromatic is a carbon ring, which contains only carbon and hydrogen having alternative double bonds existing between the carbon atoms. Aromatic rings often exhibit resonance, in which the double bonds may freely move between the carbon bonds maintaining the alternating double bound scheme. A functional group can include alcohols. Alcohols include an oxygen atom that is bonded to one hydrogen atom and one carbon atom. Alcohols can be represented by the general formula R—OH, where R is a monomer. In an embodiment, an alcohol can include a primary alcohol, secondary alcohol, or tertiary alcohol based on the carbon to which it is attached. For example, a primary alcohol is an alcohol bonded to a carbon having only one C—C bond. As a further example, a secondary alcohol is an alcohol bonded to a carbon having two C—C bonds. As a further example, a tertiary alcohol is an alcohol bonded to a carbon having three C—C bonds.

A functional group can include an ether. An ether includes an oxygen that forms single bonds with two carbon atoms of a monomer. A functional group can include a thiol. A thiol is a sulfur atom bonded to a hydrogen atom. Thiols can be represented by the general formula R—SH, where R is a monomer. Thiols can include a primary thiol, secondary thiol, or tertiary thiol, in which a primary, secondary, or tertiary thiol is based on the number of carbons that are bound to the R group of the thiol, as described above. A functional group can include an amine. An amine is a nitrogen atom bonded to a combination of carbons and hydrogens. Amines may be characterized by the formula NR₃, wherein R can be a monomer, hydrogen, carbon, oxygen, sulfur, or other atom suitable to bind to nitrogen. A primary amine includes a nitrogen bonded to one R group and two hydrogens. A secondary amine includes a nitrogen bonded to two R groups and one hydrogen. A tertiary amine includes a nitrogen bonded to three R groups and no hydrogens.

A functional group can include an aldehyde. An aldehyde is a carbon atom bonded to an oxygen atom by a double bond and at least one hydrogen by a single bond. For example, an aldehyde can include a HCHO, or RCHO, where R is a monomer. A functional group can include a ketone. A ketone is a carbon atom bonded to an oxygen atom by a double bond and bonded to at least two R groups, where the R group is a monomer. A functional group can include a carboxylic acid. A carboxylic acid is a carbon atom bonded to an alcohol group by a single bond, an oxygen atom by double bond, and a monomer by a single bond. A functional group can include an ester. An ester is a carbon that is double bonded to an oxygen, single bonded to a monomer, and single bonded to an additional oxygen that is concurrently bound to another carbon. A functional group can include an amide. An amide is a carbon that is double bonded to an oxygen, single bonded to a monomer, and single bonded to a nitrogen that is concurrently bound to hydrogen or carbon. A functional group can include a haloalkane. A haloalkane is a carbon bonded to a halogen atom, e.g., group 17 atoms such as fluorine, chlorine, bromine, iodine, or astatine.

A monomer may have one functional group or two or more functional groups. Without being bound a theory, each functional group of the two or more functional groups may have a different nucleophilicity index. For example, an alcohol functional group of a monomer may have a different nucleophilicity index compared to an ester functional group. Alternatively, an alcohol group of a monomer may have a different nucleophilicity index compared to an alternative alcohol group of the monomer.

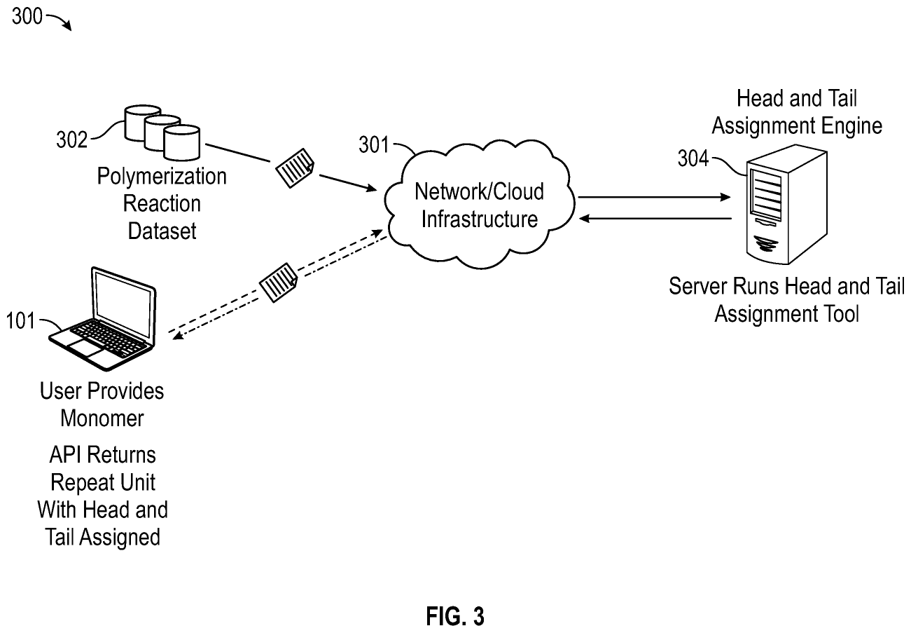

Now referring to FIG. 3, a system workflow 300 is shown. The system workflow 300 includes computer 101. A user enters a monomer or a reaction of a monomer into the computer 101 as a SMILES input. For example, a user may enter a monomer of poly(vinyl n-octyl ether) as C═COCCCCCCCC. As a further example, a user may enter a monomer of poly(vinyl n-decyl ether) as CCCCCCCCCCOC═C. As a further example, a user may enter a monomer of poly(vinylidene fluoride) as C═C(F)F. As a further example, a user may enter a monomer of poly(vinyl sec-butyl ether) as CCC(C)OC═C. As a further example, a user may enter a monomer of poly(vinylidene chloride) as C=C(Cl)Cl. As a further example, a user may enter a monomer of poly(vinyl isopropyl ether) as C═COC(C)C. Alternatively, the user may enter a polymerization reaction to form a monomer. The polymerization reaction and/or monomers are transmitted to a network/cloud infrastructure 301. The network/cloud infrastructure 301 receives a SMILES format input from a user, in which the SMILES format input is received as a csv file containing the polymer name and reaction SMILES or monomer SMILES. The SMILES format input may be based on the reaction SMILES and a polymerization reaction dataset 302. The polymerization reaction dataset 302 includes a plurality of polymerization reactions that identify one or more monomers from a plurality of polymerization reactions.

The network/cloud infrastructure transmits the monomer in SMILES format to a head and tail assignment engine 304. The head and tail assignment engine 304 runs a head and tail assignment tool to determine the location of the head and tail of the inputted SMILES format monomer. The head and tail assignment engine 304 transmits the assigned head and tail information to the network/cloud infrastructure 301, which is then sent to the computer 101 to display the repeating unit 203 of the monomer to the user. The repeating unit 203 will have the head and tail assigned on the monomer 201. The head and tail assignment allows for an accurate prediction of the polymer such that accurate polymer properties may be determined prior to synthesizing the polymer.

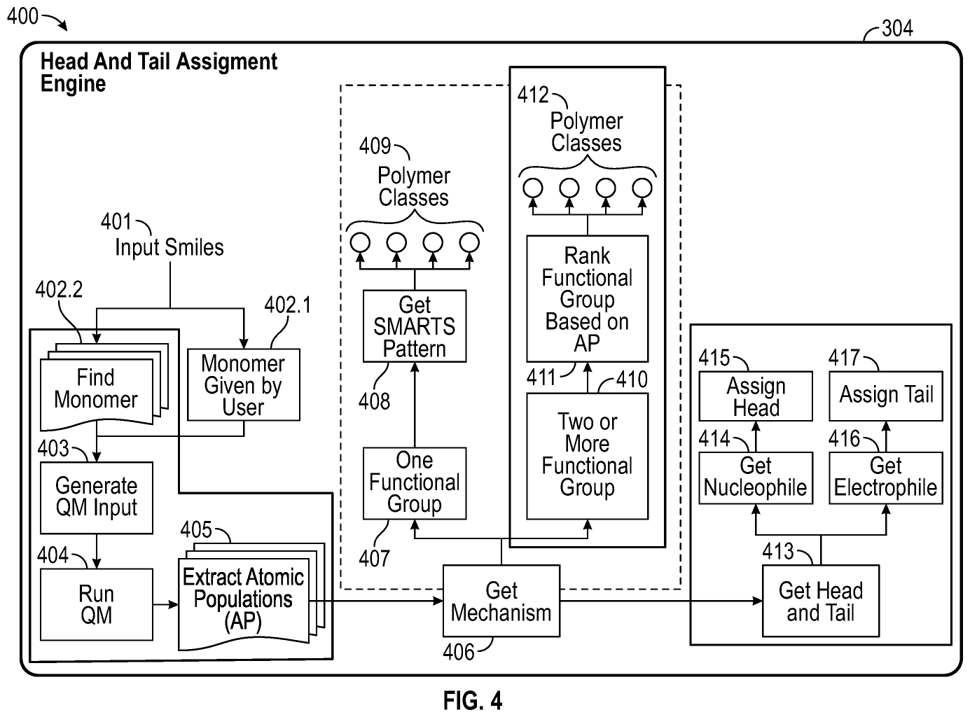

Now referring to FIG. 4, a computer-implemented method workflow 400 that is performed on a head and tail assignment engine 304 is described. At block 401, the head and tail assignment engine 304 receives a SMILES input. The SMILES input is a specification in the form of a line notation for describing the structure of chemical species using short ASCII strings. The SMILES input may be a line notation that describes either a two-dimensional drawing or three-dimensional model of the molecules and/or monomer. In an embodiment, atoms of a SMILES input are represented by a standard abbreviation of the chemical elements, in square brackets. Compounds in an organic subset, e.g., B, C, N, O, P, S, F, Cl, Br, or I do not require square brackets. Bonds are generally represented by one or more symbols such as “.”, “-”, “=”, “#”, “$”, “:”, “/”, or “\”. Rings are generally represented by a numeric label, e.g., 0, 1, 2, etc. For example, cyclohexane may be written as ClCCCCCl. Aromaticity may be represented using alternating single and double bonds, using the aromatic bond symbol, or by writing the constituent B, C, N, O, P, and S atoms in lower-case forms. Branching is described with parentheses. For example, propionic acid may be represented by the SMILES input CCC(═O)O. Stereochemistry may be characterized by the characters “/” or “\”. For example F/C=C/F may represent trans-1,2-difluoroethylene. Isotopes may be represented by a numeric symbol in brackets with the abbreviated symbol. For example, carbon-14 may be inputted as [14c].

At block 402.1, a user may enter a monomer SMILES input. For example, a user may enter a monomer smiles input of poly(vinyl n-octyl ether) as C═COCCCCCCCC. As a further example, a user may enter a monomer SMILES input of poly(vinyl n-decyl ether) as CCCCCCCCCCOC═C. As a further example, a user may enter a monomer SMILES input 402.1 of poly(vinylidene fluoride) as C=C(F)F. As a further example, a user may enter a monomer SMILES input of poly(vinyl sec-butyl ether) as CCC(C)OC═C. As a further example, a user may enter a monomer SMILES input 402.1 of poly(vinylidene chloride) as C=C(Cl)Cl. As a further example, a user may enter a monomer SMILES input 102.2 of poly(vinyl isopropyl ether) as C═COC(C)C.

Alternatively, the head and tail assignment engine 304 may, at block 402.2, find a monomer based on a polymerization reaction provided as a SMILES input. A user may enter polymerization reaction, including a plurality of reactants and a product. The head and tail assignment engine 304 may find the monomer by comparing a chemical similarity between the product and each reactant using an open source chemical similarity software. For example, the open source chemical similarity software may include RDKit. RDKit may include RDKFingerprint to generate the fingerprint of the monomer. RDKit may include FingerprintSimilarity to compare a substructure fingerprint of the product and the substructure fingerprints of each of the plurality of reactants. The fingerprints or substructure fingerprints are compared using Tanimoto Similarity until the reactant SMILES highest similarity score is selected as the monomer SMILES. The Tanimoto Similarity is described in Elementary mathematical theory of classification and prediction International Business Machines Corp., 1958), the entirety of which is incorporated herein. In an embodiment, the RDKit may set a threshold to diminish the change of selecting molecular entities with a similarity score that is too low.

Once the monomer is defined, at block 403, a coordinate file is created to generate a quantum mechanics tool input using the monomer structure. The coordinate file is a data format that specifies the coordinates and chemical element for each atom in the monomer. The coordinate file may be represented as an XYZ format, a PDB format, a mmCIF format, or an ASN.1 format. From the SMILES string the coordinate file is created using an open-source tool such as OpenBabel, with the UFF classical force field. The force field parameter includes the atomic mass, atomic charge, Lennard-Jones parameters for each atom within the monomer, the equilibrium values of bond lengths, bond angles, and dihedral angles of the bonds of the monomer. The coordinate file may represent a macromolecule or polymer, where the coordinate file can specify if an atom belongs to a standard residue or if it belongs to a hetero atom. The position of each standard residue can be specified according to the standard residue. For example, the position of a carbon atom in an amino acid may be characterized as an alpha carbon, beta carbon, gamma carbon, or delta carbon. As a further example, a nitrogen atom can be specified according to the location of the nitrogen in the standard residue, where a nitrogen in the main chain is denoted as (N), while a nitrogen on a side chain can be denoted as (NZ) due to being in the terminal zeta position of the residue. The quantum mechanics tool input defines the runtype, such as scf, basis set, such as sto3g, 631g, and maximum iterations, as any integer number (from 20 to 100, for example), necessary for the simulation to be performed on the monomer structure. For example, an input specific for the quantum chemistry tool, e.g., a General Atomic and Molecular Electronic Structure System (GAMESS-USA), for the ethylene monomer with the SMILES representation “C═C” may bre repsented by the following input:

$CONTRL SCFTYP=RHF RUNTYP=ENERGY MAXIT=30 MULT=1
$END
$SYSTEM TIMLIM=525600 MEMORY=1000000 $END
$BASIS GBASIS=STO NGAUSS=3 $END
$SCF DIRSCF=.TRUE.$END
$DATA
Title ethylene monomer
C1
C 6.0 1.06732 0.00334 0.08489
C 6.0 2.39859 0.00334 0.08489
H 1.0 0.52005 0.93711 0.16392
H 1.0 0.52005 −0.93044 0.00585
H 1.0 2.94586 0.93711 0.16392
H 1.0 2.94586 −0.93044 0.00585
$END

At block, 404 the head and tail assignment engine 304 runs the quantum mechanic tool. The quantum mechanic tool can include any software or tool capable for performing modeling using ab initio or de novo molecular quantum chemistry. In an embodiment, an open source software capable of modeling monomers using ab initio molecular quantum chemistry may be utilized to perform one or more computations of the monomer, e.g., correlation corrections, configuration interactions, second order perturbation theories, coupled-cluster approaches, density functional theory approximations, excited state approximations, nuclear gradients, vibrational frequencies, solvent effects, infinite order two component scalar relativity corrections, nuclear wavefunctions, dipole moments, frequency dependent hyperpolarizabilities, atomic overlap populations, and the like. For example, an open source software capable of computing each of these parameters can include GAMESS, from Iowa State University.

In an embodiment, the quantum mechanic tool may determine the most reactive population of electrons by determining the use of natural orbitals for atomic populations. The atomic population may be calculated based on the below equation:

$$Rx = \frac{{\sum }_{α}^{X} {\vert C α , n \vert}^{2}}{( 1 - {∈}_{n , n} )} = \frac{{\sum }_{α}^{X} {\vert C α \vert}^{2}}{( 1 - {∈}^{*} )} \qquad Eq . 1$$

where Rx is the atomic index of nucleophilicity of atom X, C α,n is the Molecular Orbital (MO) expansion coefficient of αth atomic orbital on nth MO, X is the atom index, ∈_{n,n} and ∈* are the highest occupied molecular orbital (HOMO) energy, α is the index of atomic orbital and n is the index of MO.

The head and tail assignment engine 304 extracts atomic populations from the quantum mechanics tool, at block 405. The atomic populations are extracted as a table based on electronic density data of the monomer. Electron density may be obtained according to one or more atomic overlap population methods, e.g., Mulliken, Lowdin, natural population analysis (NPA), charges from the electrostatic potential on a grid (CHELPG), Merz-Singh-Kollman (MK), atoms in molecules (AIM), or the like. In an embodiment, atomic overlap population data includes the data associated with the electronic charge distribution in a monomer and the bonding, antibonding, or nonbonding nature of the molecular orbitals for each pair of atoms of the monomer. The values that correspond to the valence orbital of each atom are extracted and organized by descending order. Without being bound by theory, the ranking of each of the atoms based on the atomic overlap population data allows identifying a predicted polymerization reaction location of the monomer 201.

Additionally, the atomic population value in an atom is directly related to the nucleophilicity index (NI) of the atom, where the higher the atomic population of the atom, the higher the nucleophilicity index, indicating the atom has a higher probability of being the polymerization site. In an embodiment, the NI of the atom may range from about 0 to about 2, in which 2 is an occupied state and 0 is an unoccupied state. Without being bound by theory this allows for the determination of the polymerization site of the polymer without unnecessary synthesis and subsequent cleavages to identify the polymerization site. For example, the calculation of the nucleophilicity index may be done where a Mulliken's scheme is used to calculate the atomic populations, in which the Mulliken's scheme characterizes the electronic charge distribution in a molecular and also the bonding, antibonding, or non-bonding nature of the molecular orbitals for pairs of atoms. The Mulliken overlap atomic populations may be calculated using ab initio quantum chemistry package GAMESS US, in which the default state functions parameters used were calculated at SCF/STO-3G theory level using the UFF force field with 5000 steps.

The head and tail assignment engine 304 determines, at block 406 the mechanism of the polymerization. The mechanism is determined by identifying at least a functional group of the monomer, where functional groups are described above. At block 407, the head and tail assignment engine 304 can determine if the monomer has one or more of a single functional group. At block 408, a SMILES arbitrary target specification (SMARTS) pattern is obtained the monomer having one or more of a single functional group. The SMARTS pattern can specify substructure patterns and atom typing of the monomer. At block 409, the head and tail assignment engine 304 can determine the one or more polymer classes that the monomer having one or more of a single functional group can exist within. For example, the one or more polymer classes can include polyvinyl polymers, polyester polymers, polyamide polymers, polyether polymers, polyurethane polymers, natural polymers, semi-synthetic polymers, synthetic polymers, linear polymers, branched-chain polymers, cross-linked polymers, addition polymerization polymers, condensation polymerization polymers, homomers, heteropolymers, co-polymers, elastomers, fibers, thermoplastics, thermosetting polymers, organic polymers, inorganic polymers, biodegradable polymers, or high-temperature polymers.

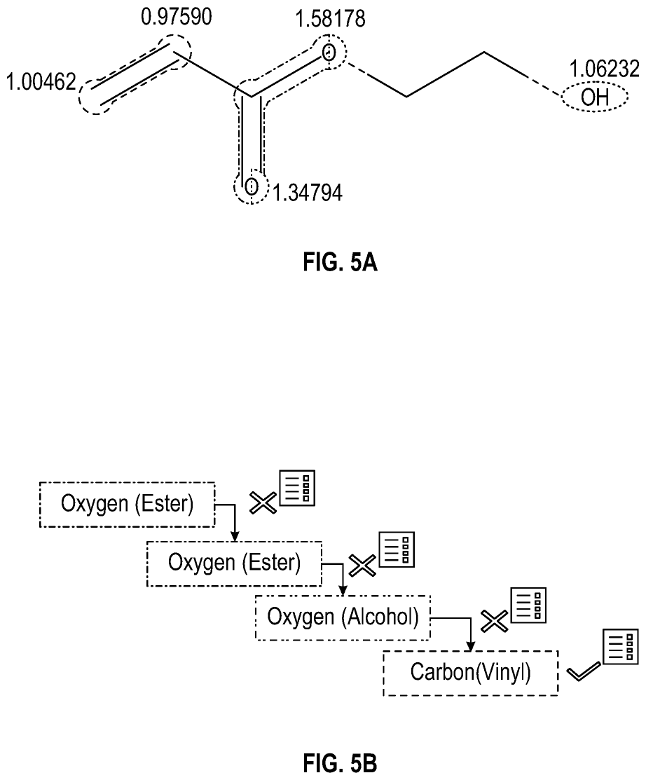

At block 410, the head and tail assignment engine 304 can determine that the monomer has two or more different functional groups by comparing the monomer to a pattern dictionary, as described herein. Now referring to FIG. 5A, a monomer may have any number of functional groups, e.g., four functional groups. For example, a monomer can have an oxygen (ester) group, an alternative oxygen (ester) group, an oxygen (alcohol) group, and a carbon (vinyl) group. The oxygen (ester) group may have a nucleophilicity index of 1.58178 1/Hartree, the alternative oxygen (ester) group may have a nucleophilicity index of 1.34794 1/Hartree, the oxygen (alcohol) group may have a nucleophilicity index of 1.06232 1/Hartree. The carbon vinyl group have may have a distal carbon having a nucleophilicity index of 0.97590 1/Hartree. The carbon vinyl group may have a proximal carbon having a nucleophilicity index of 1.00462 1/Hartree. In an embodiment, 1/Hartree may include 627 Kcal/mol. Without being bound by theory, the higher the nucleophilicity index, the higher probability the atom has of being the polymerization site.

At block 411, the mechanism is determined by producing a ranking of the functional groups based on the nucleophilicity indexes of the two or more different functional groups. The ranking occurs by comparing the atomic population of a first functional group to a second functional group, the functional group that has the higher atomic population is ranked higher due to the higher nucleophilicity index, which indicates a probability of being the polymerization site. Ranked functional groups are then compared to a pattern dictionary to determine if the functional group is a functional group that would undergo polymerization. At block 412, the head and tail assignment engine 304 determines a SMARTS pattern based on the ranking of the functional groups that can undergo polymerization, using the pattern dictionary, and determines if each functional group of the two or more functional groups exist within a polymer classes. For example, the one or more polymer classes can include polyvinyl polymers, polyester polymers, polyamide polymers, polyether polymers, polyurethane polymers, natural polymers, semi-synthetic polymers, synthetic polymers, linear polymers, branched-chain polymers, cross-linked polymers, addition polymerization polymers, condensation polymerization polymers, homomers, heteropolymers, co-polymers, elastomers, fibers, thermoplastics, thermosetting polymers, organic polymers, inorganic polymers, biodegradable polymers, or high-temperature polymers.

For example, where a functional group with a higher priority detected on a monomer is a double bond, the polymer is classified as vinylic, where the polymer class is vinyl polymerization. Additionally, the head and tail assignment engine may detect the presence of an initiator on the reaction structure. The engine may categorize the vinyl polymerization as anionic, cationic, or radical, depending on the initiator detected.

As a further example, where a functional group with a higher priority detected on a monomer is an amine, the polymer is classified as a polyamide, where the polymer class is polyamide polymerization. Polyamides can have more than one functional group involved in the reaction path, where one group is the nucleophile site and the other is the electrophile site. The head and tail assignment engine 304 will determine the nucleophile by determining the amine having the highest atomic population via a descending order, and the electrophile on the ascending order.

As a further example, if the engine finds the group amine as a probable candidate to nucleophile and alkyl halide or carboxylic acid as electrophile, the polymerization reaction is going to be categorized as a condensation reaction with polyamide class.

Now referring to FIG. 5B, a vinyl may undergo a polymerization reaction, where an ester or an alcohol would not undergo a polymerization reaction. By ranking the functional groups based on atomic population and nucleophilicity index, and subsequently eliminating the functional groups that will fail to undergo polymerization, the functional group that has the highest atomic population can be determined. This allows for the determination of the polymerization site using the atomic population of the monomer.

Referring again to FIG. 4, at block 413, the head and tail assignment engine determines the head and tail of the monomer. The head and tail of the monomer is determined based on the atomic population, the nucleophilicity index, and the specified categorizations on polymerization mechanisms. Depending on the functional groups that participate in the mechanism, the head and tail assignment engine 304 can choose an atom in the functional groups to be the head (nucleophile) and the tail (electrophile). At block 414, the head and tail assignment engine 304 determines a nucleophile based on the atomic population and the nucleophilicity index, where the nucleophile is the atom in functional group that has the higher nucleophilicity index. At block 415, the head and tail assignment engine 304 then assigns the nucleophile as the head of the polymerization reaction site.

At block 416, the head and tail assignment engine 304 determines an electrophile where the electrophile is the atom in functional group that has the lower nucleophilicity index and/or is determined according to the polymer class defined. At block 417, the head and tail assignment engine 304 then assigns the electrophile as the tail of the polymerization reaction site.

The head and tail assignment engine tags the molecule with the head and tail and sanitizes the SMILES string by deleting atom mappings and unnecessary tokens such as double bonds on vinylic polymers. The head and tail assignment engine 304 then outputs a comma-separated value (csv) table, with the name of the polymer, the full polymerization reaction, the defined monomer, the mechanism and the monomer smiles tagged with head and tail atoms.

Overall, the head and tail assignment engine 304 receives a polymer smiles (the pseudo repeating unit) from a reaction smiles as an input smiles at block 401, and is analyzed under chemical similarity comparison to find the probable monomer unit on the reactants at block 402.2. The user may also provide the already defined monomer structure at block 402.1. The monomer SMILES is used to generate the quantum mechanics tool input (103) which is run by the quantum mechanics tool. The atomic overlap populations are extracted from the quantum mechanics tool results and organized by descending order, in which atomic population values are used to get the polymerization mechanism. If the monomer smiles has only one functional group, a SMARTS pattern is acquired to classify the polymerization mechanism. If the monomer smiles has two or more functional groups, the functional group related to a polymerization mechanism with higher atomic population is going to be selected to classify the mechanism. Without being bound by theory, the usage of the quantum mechanics tool run with functional group improves the accuracy of the determination of the monomer head and tail. From the mechanism, the head and tail are assigned. The head is assigned by getting the nucleophile from the functional group related to a polymerization mechanism with the higher atomic population and the tail is assigned by getting the electrophile from the functional group related to the polymerization mechanism with the lower atomic population. Without being bound by theory, the assignment of the head and the tail of the monomer allows for an accurate prediction of the polymerization of one or more monomers and a reduction in unnecessary complex synthesis of polymers is achieved, which reduces the overall cost of production as well as time spent on identifying target polymers.

### Examples

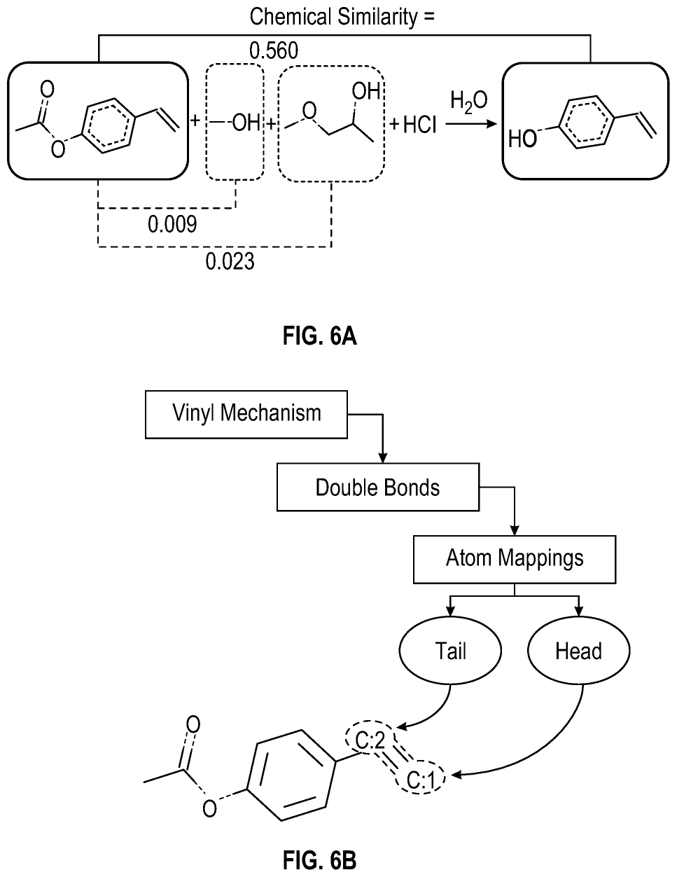

Now referring to FIG. 6, a computer-implemented method workflow of determining a p-hydroxystyrene monomer is shown. In an embodiment, the polymerization reaction of p-hydroxystyrene is analyzed to obtain a reaction monomer based on chemical similarity using RDKFingerprint, as shown in FIG. 6A. The chemical similarity of p-hydroxystyrene was found to be 56% similar to 4-acetoxystyrene, where the chemical similarity identifies a percentage of similarity of chemical elements, molecules, or chemical compounds with respect to either structure or functional qualities of the two structures. Moreover, the chemical similarity between the remaining reactants, e.g., methanol and 1-methoxy-2-propanol was found to be 0.9% and 2.3%, respectively. The compound with the higher similarity was determined to be the reaction monomer, in which 4-acetoxystyrene was the reaction monomer for the polymerization reaction of p-hydroxystyrene. A quantum mechanics tool input was then generated based on the structure of 4-acetoxystrene. The input included the runtype, such as scf or opt, basis set, such as sto3g, 631g and steps, such as any integer number.

The quantum mechanics tool was then simulated and Mulliken atomic overlap populations were extracted from a log file obtained by the quantum mechanics simulation. The values corresponding to the valence orbitals of atoms were extracted. The values were then assigned to each of the functional groups located on the 4-acetoxystyrene. The functional groups located on the 4-acetoxystyrene were identified, in which an ester group and a vinyl group were determined. Even though the ester group had a higher atomic population, based on the pattern dictionary, the ester is not related to any polymerization reaction and would not undergo a polymerization reaction. The vinyl group had a lower atomic population and was found to be able to undergo a polymerization reaction. As such, the polymerization site was determined to be the vinyl group, as shown in FIG. 6B.

The head and tail were then assigned to the vinyl group, in which the carbons were labeled C1 and C2. The head position was labeled as the nucleophilic carbon. The tail position was labeled as the electrophilic carbon. As such, the head was assigned to be the C1 position, while the tail was assigned to be the C2 position, as shown in FIG. 6B. Without being bound by theory, where there is no information regarding the reaction initiator, the head and tail may be randomly assigned.

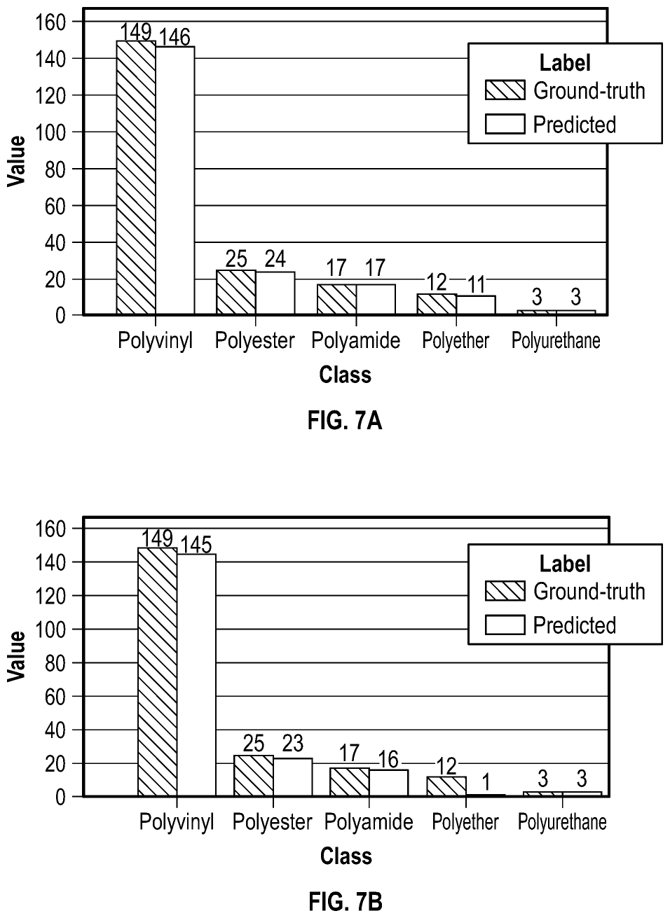

Now referring to FIGS. 7A and 7B, validation of the performance of the head and tail assignment engine was performed. To validate the performance of HTA, a dataset with 206 polymer precursors that belongs to polyamide, polyester, polyether, polyurethane class and also undergo through vinyl polymerization were analyzed. From the 206 polymer precursors, the head and tail assignment engine correctly predicted the class of 201 monomers including polyvinyl polymers, polyester polymers, polyamide polymers, polyether polymers, and polyurethane polymers, which represents 97.6% of accuracy, as shown in FIG. 7A.

Regarding the head and tail assignment, the algorithm could correctly assign the head and tail positions for 188 monomers, where the monomers were polyvinyl polymers, polyester polymers, polyamide polymers, polyether polymers, or polyurethane polymers, which translates to 91.3% of accuracy, as shown in FIG. 7B.

Overall, the present disclosure provides a computer-implemented method of identifying a head and tail of a monomer such that accurate polymer properties are capable of being predicted. A monomer can be utilized, e.g., an IUPAC named monomer, an unknown monomer, or unnamed monomer, as the only requirement of the monomer is the SMILES format, allowing for a wide range of monomers to be utilized, allowing for a broad range of compounds to be used. Using an atomic population of the monomer allows for a nucleophilicity index of each atom of the monomer to be determined, which creates improved identification of the head and tail of the monomer, allowing for improved polymer property predictions. The atoms are ranked using the nucleophilicity index, allowing for the head and tail of a monomer having two or more functional groups to be identified, which reduces overall error in modeling of the polymer. By incorporating head and tail characterization of monomers a reduction in unnecessary complex synthesis of polymers is achieved, which reduces the overall cost of production as well as time spent on identifying target polymers.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the foregoing is directed to embodiments of the present disclosure, other and further embodiments of the disclosure may be devised without departing from the basic scope thereof, and the scope thereof is determined by the claims that follow.

What is claimed is:

1. A computer implemented method comprising:
obtaining an input, wherein the input comprises a simplified molecular input line entry;
generating a quantum mechanics tool based on the simplified molecular input line entry;
extracting an atomic population of a monomer using the quantum mechanics tool;
identifying a functional group of the monomer;
determining a polymerization site based on the atomic population of the monomer and the functional group; and
assigning a head and a tail of the polymerization site of the monomer.

2. The method of claim 1, wherein the input comprises a polymerization reaction.

3. The method of claim 2, further comprising determining the monomer based on the input by comparing a product and a reactant of the polymerization reaction.

4. The method of claim 1, further comprising determining a nucleophilicity index of each atom of the monomer using the atomic population of the monomer.

5. The method of claim 4, further comprising identifying a plurality of functional groups of the monomer.

6. The method of claim 5, further comprising ranking the functional groups based on the nucleophilicity index of each functional group.

7. The method of claim 1, further comprising obtaining a functional group from a pattern dictionary and identifying the functional group of the monomer based on the pattern dictionary.

8. The method of claim 7, wherein the pattern dictionary comprises a plurality of polymerization classes.

9. The method of claim 8, wherein the polymerization classes comprise a vinyl polymerization or polyamide polymerization.

10. The method of claim 1, further comprising:
identifying a nucleophile of the polymerization site;
identifying an electrophile of the polymerization site; and
assigning the head and tail of the polymerization site based on the nucleophile and the electrophile, wherein the nucleophile is the head and the electrophile is the tail.

11. A system, comprising:
a computer;
a polymerization dataset;
a network; and
a server, wherein the server is configured to:
obtain an input, wherein the input comprises a simplified molecular input line entry;
generate a quantum mechanics tool based on the simplified molecular input line entry;
extract an atomic population of a monomer using the quantum mechanics tool;
identify a functional group of the monomer;
determine a polymerization site based on the atomic population of the monomer and the functional group; and
assign a head and tail of the polymerization site of the monomer.

12. The system of claim 11, wherein the input is a polymerization reaction and the server is further configured to determine a monomer based on the input by comparing a product and a reactant of the polymerization reaction.

13. The system of claim 11, wherein the server is further configured to determine a nucleophilicity index of each atom of the monomer using the atomic population of the monomer.

14. The system of claim 13, wherein the server is further configured to identify a plurality of functional groups of the monomer.

15. The system of claim 14, wherein the server is further configured to rank the functional groups based on the nucleophilicity index of each functional group.

16. The system of claim 11, wherein the server is further configured to:
obtain a functional group from a pattern dictionary; and
identify the functional group of the monomer based on the pattern dictionary.

17. A computer program product for assigning a head and tail of a monomer, the computer program product comprising:
a computer-readable storage medium having computer-readable program code embodied therewith, the computer-readable program code executable by one or more computer processors to:
obtain an input, wherein the input comprises a simplified molecular input line entry;
generate a quantum mechanics tool based on the simplified molecular input line entry;
extract an atomic population of a monomer using the quantum mechanics tool;
identify a functional group of the monomer;
determine a polymerization site based on the atomic population of the monomer and the functional group; and
assign a head and a tail of the polymerization site of the monomer.

18. The computer program product of claim 17, wherein the input is a polymerization reaction and the computer-readable program code is further executable to determine the monomer based on the input by comparing a product and a reactant of the polymerization reaction.

19. The computer program product of claim 17, wherein the computer-readable program code is further executable to:
determine a nucleophilicity index of each atom of the monomer using the atomic population of the monomer;
identify a plurality of functional groups of the monomer; and
rank the functional groups based on the nucleophilicity index of each functional group.

20. The computer program product of claim 17, wherein the computer-readable program code is further executable to:
obtain a functional group from a pattern dictionary; and
identify the functional group of the monomer based on the pattern dictionary.
